(19) United States
(12) Patent Application Publication
(10) Pub. No.: US 2008/0026065 A1
(43) Pub. Date: Jan. 31, 2008

(54) Powdered Compositions Of Sensitive Active Materials In An At Least Partially Amorphous State

(76) Inventors: Gerald Adams, Wiltshire (GB); Peter Alan Lambert, Surrey (GB); Valerie Adams, Wilts. (GB)

Correspondence Address: HOFFMANN & BARON, LLP, 6900 JERICHO TURNPIKE, SYOSSET, NY, 11791, US

(21) Appl. No.: 10/591,369

(22) PCT Filed: Feb. 28, 2005
(86) PCT No.: PCT/GB05/00742

§ 371 Date: Aug. 15, 2007

(30) Foreign Application Priority Data
Mar. 1, 2004 (GB) 0404586.0

(51) Int. Cl. A61K 9/14; A61K 31/715; A61K 35/00; A61K 35/74; A61K 35/76; A61K 36/06; A61K 38/02; A61K 38/43; A61K 47/36

(52) U.S. Cl. 424/489; 424/195.15; 424/93.1; 424/93.4; 424/93.6; 424/94.1; 514/2; 514/54; 514/772; 514/777

(57) ABSTRACT

The invention provides a powdered formulation which is a freeze-dried mixture of a sensitive active material and an excipient containing: 0.1-50% by wt of the sensitive active material, 50-99.99% by wt of the excipient, wherein at least 0.1% by wt of the mixture is an amorphous state; the formulation has a substantially reduced hygroscopicity.

The present invention provides a stable formulation of a sensitive active material which has reduced hygroscopicity and a process for preparing such a formulation particularly by lyophilisation.

Lyophilisation (freeze-drying) and drying techniques are well known as methods for stabilising a sensitive active material. A sensitive active material is generally understood to be a labile active material and includes a sensitive organic and/or inorganic molecule, a biopolymer, for example a polypeptide, protein, enzyme, hormone, vitamin, antibiotic, polysaccharide, lipid, killed or live whole live cell, including a virus (including phage), bacterium, fungus and/or eukaryote. Such an active material may be used as a vaccine, a starter organism for the brewing, baking, composting, and/or silage production, the industrial production of a solvent, antibiotic and/or bioproduct etc.

A pharmaceutical product, live cell or a bioproduct thereof, presented for lyophilisation or drying, should be formulated to ensure that the dried product has one or more of the following characteristics:

Active;

Shelf stable;

Cohesive as plug or cake;

Dried to a prescribed moisture content;

Cosmetically presentable for pharmaceutical and/or commercial acceptability; and

Soluble (preferably readily soluble).

In addition, when the dried product is intended to be disseminated or used in the powder form, it may be essential to induce a discreet particle size which is either pharmaceutically efficacious or which prevents the powder from coalescing and occluding the aerosol, spray device, ejector or inhaler. If required, the dried powder should be capable of withstanding further processing such as de-aggregation, mixing, milling, dispensing and/or packaging etc.

To improve these characteristics, an additive and/or excipient may be included in the formulation in combination with the active component.

Physically individual constituents may be:

Wholly crystalline;

Wholly amorphous; or

Initially crystalline but converted to the amorphous form by the drying or lyophilisation process.

It is recognised that the persistence of the amorphous state may be a prerequisite when particular sensitive active materials (for example inorganic or organic molecules or bioproducts, including polypeptides, proteins, enzymes, killed whole or live cells) are dried. This is because such a labile active material may require specific protection by an excipient at the molecular level in order to be stabilised.

However the persistence of an amorphous state after drying generally results in the absorption of moisture and air into the dried product if the powder is exposed to the atmosphere. This will degrade the product and/or make it difficult to administer.

To prevent the ingress of moisture and air, it is necessary to seal the product in a container within the lyophiliser or dryer prior to removal of the product or to unload the product from the lyophiliser or dryer into an inert, moisture free atmosphere and ensure all additional processing is carried out in such an environment. Such arrangements add expense to the process for the preparation of the product.

Generally the usual containers for an inhaler, ejector, flask, sachet, tablet or oral dose will allow a small degree of moist air ingression. For a sensitive active material this ingression may be sufficient for product activity or shelf stability to be compromised. Where the product is in the form of a dry powder, it is also possible that the product may coalesce sufficiently to block an ejector or inhaler thereby adversely affecting its efficiency.

Specific adverse effects of moisture or air absorption include:

A change in the particle size resulting from moisture absorption into the powder which is intended to be disseminated as a discrete particle from the ejector or inhaler a powder aerosol for nasal or pulmonary application or a change in the particle size and physical characteristics if the powder intended to be disseminated by a shaker or ejector for oral, aural, topical (that is application to a wound, internal organ or skin), ophthalmic or anal application, such that the efficiency of the ejector or applicator is compromised.

The exposure or increase in moisture content of the powder above an optimum may also reduce shelf stability by encouraging chemical degradation of the product resulting in reduced shelf stability.

As well as altering the powder size distribution and physical properties by moisture absorption outlined above, reactive atmosphere gases, such as oxygen or carbon dioxide entering powder product may affect the activity, efficacy or shelf stability of the pharmaceutical or product intended to be administered as a powder, distributed as a composting or starter culture or dried product intended to be reconstituted for injection or administration.

A solution to these problems has been sought.

According to the invention there is provided a powdered formulation which is a freeze-dried mixture of a sensitive active material and an excipient containing:

from 0.01 preferably from 0.1, more preferably from 0.5 to 50% by wt of the sensitive active material,

from 50 to 99.99, preferably to 99.9, more preferably to 99.5% by wt of the excipient,

wherein at least 0.1% by wt of the mixture is an amorphous state.

It has surprisingly been found that by combining the sensitive active material and excipient of the formulation into a stable crystalline/amorphous matrix, the formulation has substantially reduced hygroscopicity. The hygroscopicity of the formulation according to the invention measured by the percentage increase in the weight of the formulation after 8 hours in a 75% relative humidity environment is preferably less than 5% by weight, more preferably less than 3% by weight, most preferably less than 2% by weight. Thus the invention eliminates the need to protect the dried product after its removal from the lyophiliser or dryer for milling and final packaging when the powdered formulation is exposed to the atmosphere or to prevent the ingress of moist air into the product during storage or dissemination of the formulation in its primary container e.g. aerosol spray or ejector.

According to the invention there is further provided a dosage form comprising the formulation according to the invention. The dosage form may optionally be a container which comprises the formulation (such as a capsule (particularly a gelatine capsule), an oral dose container, flask or sachet) or an article (such as a tablet) which has been formed from the formulation.

According to the invention there is also provided a pharmaceutical formulation according to the invention for use in therapeutic treatment of a human or animal body by nasal administration.

According to the invention there is further provided use of a formulation according to the invention in the manufacture of a medicament for use in therapeutic treatment of a human or animal body by nasal administration.

In the formulation of the invention, suitably from 0.1, preferably from 0.5, more preferably from 1 to 50% by wt of the mixture is in an amorphous state.

For example one formulation of the invention may contain:

from 0.01, preferably from 0.1, more preferably from 0.5 to 50% by wt of sensitive active material in amorphous state,

from 50 to 99.99, preferably to 99.9, more preferably to 99.5% by wt of excipient in crystalline state,

0-5% by wt of excipient in amorphous state.

For example another formulation of the invention may contain:

from 0.01, preferably from 0.1, more preferably from 0.5 to 50% by wt of sensitive active material in crystalline state,

from 50 to 99.89, preferably to 99.8, more preferably to 99.4% by wt of excipient in crystalline state,

0.1-5% by wt of excipient in amorphous state.

More specifically a formulation of the invention may contain:

from 0.01, preferably from 0.1, more preferably from 0.5 to 25% by wt of amorphous or crystalline state of sensitive active material,

from 75 to 99.49, preferably to 99.4, more preferably to 99% by wt of crystalline state excipient,

0.5-5% by wt of amorphous state excipient.

A small (from 0.1 to 10% w/w, preferably from 0.1 to 1% w/w) amount of an additive/stabilizer for the sensitive active material (such as an antioxidant, free radical scavenger and/or a Maillard reaction suppresser) may be included if desired. An antioxidant, free radical scavenger and/or a Maillard reaction suppresser is useful to prevent loss of shelf stability as a result of oxidation, the induction of free radicals or Maillard reactions induced by the drying process.

The freeze dried mixture of excipient and sensitive active material used in the invention is generally in composition identical to that of an aqueous solution of the excipient and the sensitive active material. Therefore an average particle of a formulation according to the invention will contain sensitive active material and excipient in the same percentage amounts as their percentages in the original solution.

The crystalline/amorphous character of the sensitive active material and excipient intended for freeze-drying in accordance with this invention may be assessed as three groups:

1. Active material(s) and excipient(s) which are crystalline and which persist in this form throughout freeze drying to provide a dried, crystalline matrix. The excipient(s) may be defined as crystalline and may be selected from a eutectic salt (such as sodium chloride, potassium chloride), certain amino acids (such as glycine), certain sugar alcohols (such as mannitol and sorbitol), and other organic molecules.

2. Active material(s) and excipient(s) which are crystalline but which may be induced into a non-crystalline or amorphous state by freezing and once in this state remain amorphous throughout subsequent drying, final finishing, storage and distribution. Examples include certain amino acids (such as glutamine or serine), a monosaccharide (such as glucose), a disaccharide (such as sucrose, trehalose, lactose), a trisaccharide (such as raffinose), a polysaccharide, certain polyethylene glycols (such as polyethylene glycols having a molecular weight of about 6000), certain polypeptides (such as a polyamino acids) and/or polymers (such as poly-d-lactic acid).

3. Active material(s) and/or excipient(s) which are non-crystalline (amorphous) and which are maintained in the amorphous state throughout subsequent drying, dispensing and final finishing, storage and distribution. Examples include certain saccharides (such as amorphous lactose), certain polyethylene glycols (such as polyethylene glycols having a molecular weight up to 1000), a polyglycan, a polysaccharide (such as a dextran), a cyclodextrin, povidone, micro-fine cellulose, certain polymers (such as potato starch) and a protein.

Compounds in groups 2 and 3 are defined as amorphous for the purposes of the present invention.

The sensitive active material is preferably a labile active material, especially a labile organic and/or inorganic molecule, a biopolymer, a polypeptide, protein, enzyme, hormone, vitamin, antibiotic, polysaccharide, lipid, killed or live whole live cell (especially a virus (including a phage), bacterium, fungus and/or eukaryote). A labile material is generally understood to be a material which is subject to degradation under normal conditions (i.e. ambient temperature, pressure and humidity) or which is chemically reactive or unstable under normal conditions.

The sensitive active material is preferably an enzyme (such as lactic dehydrogenase, L-asparaginase, or phenylalanine ammonia lyase), a live yeast for seed culture (such as Saccharomyces cerevissiae), a live bacterium for seed culture (such as Escherichia coli), live bacterium for diagnostic use (such as Salmonella typhimurium), a live bacterium for silaging use (such as Lactobacillus acidophilus), a live, attenuated vaccine (such as influenza virus strain WSN), or a phage for therapeutic or diagnostic use (such as phage (p174).

Several sensitive active materials may be incorporated into a single formulation to provide a more effective product. The sensitive active material may be augmented by adding a simple or complex compound which may not be active itself but which may potentiate the effects of the active component(s) or act as an adjuvant.

An excipient should preferably satisfy one or more of the following parameters:

Be compatible with processing requirements;

Be non-damaging to the active material;

Provide a soluble, absorbable product;

Provide a shelf-stable product; and

Provide a commercially acceptable product.

The formulation according to the invention is preferably a pharmaceutical formulation. Where the formulation is a pharmaceutical formulation, an excipient preferably should satisfy one or more of the following parameters:

Be pharmacologically inert; and

Be well tolerated by animal or human tissue (especially nasal tissue and nasal physiological function or aural tissue and aural physiological function or oral tissue or oral physiological function or opthalmic tissue or opthalmic physiological function or wound/target tissue or wound/target tissue physiological function or anal/alimentary tract tissue or anal/alimentary tract physiological function).

An excipient that may be used includes a saccharide, a polysaccharide and/or a sugar alcohol.

The term “cyclodextrin” refers to a cyclic oligosaccharide, such as alpha-, beta- and gamma-cyclodextrin and/or a derivative thereof, such as methylated beta-cyclodextrin.

The term “saccharide” includes a monosaccharide (such as glucose), a disaccharide (such as lactose, maltose, trehalose, sucrose, and/or saccharose) and a polysaccharide (such as a dextran).

The term “sugar alcohol” refers to a saccharide polyol such as mannitol, sorbitol, inositol and/or xylitol.

The formulation according to the invention has the advantage that no preservatives (i.e. bactericides or fungicides) are necessary.

The pharmaceutical formulation according to the invention may be administered parenterally after reconstitution in a sterile fluid or used or applied as a solution or suspension after reconstituting in a sterile or non-sterile reconstitution fluid.

The pharmaceutical formulation according to the invention can also be administered using a nasal insufflator or a passive device. For example, the formulation is placed in a capsule which is set in an inhalation or insufflation device. A needle is penetrated through the capsule to make pores at the top and the bottom of the capsule and air is drawn in by inhalation or blown through the device to force out the powder particles into the patient's nose. The formulation can also be administered in a jet-spray of an inert gas or suspended in liquid organic fluids. The required amount for a nasal administration of a formulation according to the invention may be, for example, between 1 and 50 mg, typically 1 to 20 mg, for example administered as about 5 to 20 mg per nostril.

The formulation of the present invention is generally prepared by freeze-drying. The sensitive active material and the excipient should be compatible with the drying process and should provide a bulk within the processing container to prevent the migration of drying product during processing (ablation).

A further advantage of the formulation according to the invention is that it is possible to predictably obtain a resultant dried powder which exhibits a particle size suitable for comfortable retention and a fast dissolution of the active material in the nasal mucosa, followed by absorption into the systemic circulation. The formulation according to the invention comprises particles which remain stable and uniform throughout processing, final finishing, storage and distribution. The formulation is shelf-stable and free-flowing, presents no problems when dispensed into its final container and is simple to administer by the patient.

The ratio and persistence of the amorphous and crystalline contents of the formulation according to the invention may be determined for compliance with crystalline/amorphous parameter defined above by a thermal analysis technique including differential scanning calorimetry. The particle size distribution pattern of the formulation may be defined by particle size characterisation using a laser diffraction technique with, for example, Mastersizer instrumentation from Malvern Instruments. This laser diffraction powder characterization technique may be carried out directly on a dry powder sample of the formulation (dry analysis) or on a sample of the formulation suspended in a solvent in which the formulation is not soluble (wet analysis). It is necessary to ensure that each sample analysed is fully de-aggregated at the time of characterization and this is best achieved using the wet analysis method. With this method de-aggregation of particle agglomerates can be achieved by the use of dispersing agents, surfactants and/or sonication of the sample prior to analysis and maintained by stirring or recirculation of the sample during analysis. In addition, de-aggregation of the sample can be verified visually under a microscope.

By complying with the crystalline/amorphous parameter defined above and provided that an aqueous formulation of the components is compatible with freeze drying, then the formulation according to the invention preferably has one or more of the following properties:

A particle size suitable for nasal delivery that can be induced and maintained by freeze-drying;

The small particle size distribution (finings) wherein small particles generally have a size less than 5 μm, is minimized;

When removed from the freeze-dryer and exposed to atmosphere, the particles of the formulation do not alter in size nor absorb moisture to the extent that the particles aggregate or become sticky, thereby preventing final finishing or dispensing and also influencing pharmacological activity;

The resultant nasal powder exhibits high solubility, improved nasal absorption and, as a consequence, very high pharmacological or biological activity.

According to the invention there is also provided a method of medical treatment which method comprises supplying to a human or animal (preferably mammal) patient a therapeutically effective amount of a formulation according to the invention or a therapeutically effective amount of a dosage form according to the invention.

According to the invention there is also provided a method of preparing a powdered formulation which comprises forming a mixed solution of active material and excipient(s) containing:

from 0.01 preferably from 0.1, more preferably from 0.5 to 50% by wt of the sensitive active material,

from 50 to 99.99, preferably to 99.9, more preferably to 99.5% by wt of the excipient,

and freeze-drying the solution so that at least 0.1% by wt of the freeze-dried blend is in an amorphous state.

In the method of the invention, freezing conditions should preferably be selected to provide:

An optimal ice crystal structure conducive to maximal sublimation rate;

The maintenance of a crystalline phase within the matrix; and/or

The induction of and/or maintenance of an amorphous phase within the matrix.

Selection of suitable freezing conditions will be influenced by

The chemical nature and concentration of the sensitive active material and crystallising or amorphous excipient within the solution or suspension;

Freeze dryer design and specification;

Primary container used to process the product; and/or

Sample fill depth.

Differential scanning calorimetry, differential thermal analysis and resistance analysis may be used to define optimum freezing conditions. From such analysis, we have found it desirable that the product should be frozen at a slow rate or a heat annealing cycle applied to induce or maintain the correct matrix composition. For example a freezing rate of about 0.1 to 0.5° C. per minute and a heat annealing cycle comprising, for example: cool product to −45° C. at 0.1-1.0° C. per minute; hold 2 hours, warm to −15° C., hold 2 hours, re-cool to −45° C., hold 2 hours before drying; have been used. These values may be used for guidance, but will vary depending on the formulation of the active material and limitations introduced by the apparatus and other component(s) used in freeze-drying.

For example a suitable drying cycle includes heating directly to 5° C. for main drying, increased chamber pressure to 150 mTorr to facilitate heat input and increased final drying temperature to 20° C. Variations on this cycle, designed for specific product/process optimization include a cycle where shelf temperature was raised to 15° C. for the initial phase of main (primary) drying and then progressively reduced to 5° C. for the remainder of main (primary) drying with chamber pressure increased up to 300 mTorr to facilitate heat input into product followed by increased shelf temperature to 25° C. for final (secondary) drying.

Factors which determine the freeze-drying characteristics of a sample include:

The glass transition temperature (Tg′) which determines the temperature at which the viscosity of the cooled mass decreases sufficiently so that the sample collapses during freeze-drying. Glass/transition temperatures have been determined by differential scanning calorimetry, differential thermal analysis and resistance analysis;

Operationally the temperature at which sample collapses during freeze-drying is defined as the collapse temperature (Tc). Collapse temperatures are determined by freeze-drying microscopy. In the absence of complicating factors such as the development of surface skins on the drying sample, collapse and glass transition temperatures are typically similar;

Skin formation and associated defects, are also determined by freeze-drying microscopy.

The invention is illustrated by way of example with reference to the Figure of the accompanying drawings which shows a graph showing the percentage increase in weight for freeze dried samples of mannitol and trehalose exposed to a 75% relative humidity environment.

The following Examples which illustrate the invention are not intended to limit the scope of the claims.

### METHOD EXAMPLE 1

The following freeze-drying cycle has been used effectively for formulations having Tg′ or Tc at c. −16 to −18° C. This means that the product temperature should be maintained at c. -−23° C. (i.e. −18° C. plus 5° C. for operational safety=−23° C.).

Freeze to −445° C.

Cooling rate 0.25° C. per minute

Hold 120 minutes

### Main Drying:

Shelf Temperature (step 1) −20° C.

Warming rate 1.0° C. per minute

Hold 1200 minutes

Chamber pressure 50 mTorr

### Shelf Temperature (step 2) 0° C.

Warming rate 1.0° C. per minute

Hold 720 minutes

Chamber pressure 50 mTorr

### Shelf Temperature (step 3) 5° C.

Warming rate 1.0° C. per minute

Hold 1000 minutes

Chamber pressure 50 mTorr

Final Drying

### Shelf Temperature 15° C.

Warming rate 1.0° C. per minute

Hold 700 minutes

Chamber pressure 50 mTorr

### EXAMPLES 2 AND 3

The activity of the enzyme lactic dehydrogenase after freeze drying was measured for formulations according to the invention and for a comparative formulation. Measurement of activity was carried out chemically by the reaction of the enzyme with its reactant, lactose.

The formulations set out below were prepared using the freeze drying cycle set out in Method Example 1:

### Comparative Example 1

0.1 to 1.5 g enzyme

2.0 g Mannitol

Made up to 100 g water as start formulation before freeze-drying

### Example 2

0.1 to 1.5 g enzyme

2.0 g Mannitol

1.0 g Lactose (amorphous after freeze drying)

Made up to 100 g water as start formulation before freeze-drying

### Example 3

0.1 to 1.5 g enzyme

2.0 g Mannitol

1.0 g Glucose (amorphous after freeze drying) 2.0 g Dextran (mw 70,000 amorphous after freeze-drying)

Made up to 100 g water as start formulation before freeze-drying

It was found that the enzyme in Comparative Example 1 had 8% activity after the freeze drying process. In comparison the enzyme of Examples 2 and 3 according to the invention had an activity after lyophilisation of 60%.

### EXAMPLES 4 TO 7

The activity of enzyme L-asparaginase (which is a known anticancer drug) after freeze drying was measured for formulations according to the invention and for a comparative formulation. Measurement of activity was carried out chemically by the reaction of the enzyme with its reactant, L-asparagine.

The formulations set out below were prepared using the freeze drying cycle set out in Method Example 1:

### Comparative Example 2

0.1 to 1.5 g enzyme

2.0 g Mannitol

Made up to 100 g water as start formulation before freeze-drying

### Example 4

0.1 to 1.5 g enzyme

2.0 g Mannitol

1.0 g Glucose (amorphous after freeze-drying)

Made up to 100 g water as start formulation before freeze-drying

### Example 5

0.1 to 1.5 g enzyme

2.0 g Mannitol

1.0 g Lactose (amorphous after freeze-drying)

Made up to 100 g water as start formulation before freeze-drying

### Example 6

0.1 to 1.5 g enzyme

2.0 g Mannitol

1.0 g Sucrose (amorphous after freeze-drying)

Made up to 100 g water as start formulation before freeze-drying

### Example 7

0.1 to 1.5 g enzyme

2.0 g Mannitol

1.0 g Trehalose (amorphous after freeze-drying)

Made up to 100 g water as start formulation before freeze-drying

It was found that the enzyme in Comparative Example 2 had less than 1% activity after the freeze drying process. In comparison the enzyme of Examples 4 to 7 according to the invention had an activity after lyophilisation of 100%.

### EXAMPLE 8

The activity of enzyme phenylalanine ammonia lyase (a known pharmaceutical agent) after freeze drying was measured for a formulation according to the invention and for a comparative formulation. Measurement of activity was carried out chemically by the reaction of the enzyme with its reactant, phenylalanine.

The formulations set out below were prepared using the freeze drying cycle set out in Method Example 1:

### Comparative Example 3

0.1 to 1.0 g enzyme

1.0 g Mannitol

Made up to 100 g water as start formulation before freeze-drying

### Example 8

0.1 to 1.5 g enzyme

2.0 g Mannitol

1.0 g Lactose (amorphous after freeze-drying)

Made up to 100 g water as start formulation before freeze-drying

It was found that the enzyme in Comparative Example 3 had less than 5% activity after the freeze drying process. In comparison the enzyme of Example 8 according to the invention had an activity after lyophilisation of 70%.

### EXAMPLES 9 AND 10

The viability of Saccharomyces cerevissiae (live yeast for seed culture) after freeze drying was measured for formulations according to the invention and for a comparative formulation. Measurement of viability was by titres expressed as the number of colony forming units (cfu) per ml of fungal suspension as plated using solid agar plates.

The formulations set out below were prepared using the freeze drying cycle set out in Method Example 1:

### Comparative Example 4

10⁸˜10¹² colony forming units Saccharomyces cerevissiae

Made up to 100 g water as start formulation before freeze-drying

### Example 9

10⁸˜10¹² colony forming units Saccharomyces cerevissiae

10 g Mannitol

1.0 gm Trehalose (amorphous after freeze-drying)

Made up to 100 g water as start formulation before freeze-drying

### Example 10

10⁸˜10¹² colony forming units Saccharomyces cerevissiae

10 g Mannitol

1.0 gm Glucose (amorphous after freeze-drying)

Made up to 100 g water as start formulation before freeze-drying

It was found that Saccharomyces cerevissiae in Comparative Example 4 had less than 1% viability after the freeze drying process. In comparison Saccharomyces cerevissiae of Examples 9 and 10 according to the invention had a viability after lyophilisation of 25%.

### EXAMPLE 11 TO 15

The viability of Escherichia coli (live bacterium for seed culture) after freeze drying was measured for formulations according to the invention and for a comparative formulation. Measurement of viability was by titres expressed as the number of colony forming units (cfu) per ml of bacterial suspension as plated using solid agar plates.

The formulations set out below were prepared using the freeze drying cycle set out in Method Example 1:

### Comparative Example 5

10⁶˜10¹² colony forming units Escherichia coli

1.0-5.0 g Mannitol

Made up to 100 g water as start formulation before freeze-drying

### Example 11

10⁶˜10¹² colony forming units Escherichia coli

1.0-5.0 g Mannitol

1.0 g Trehalose (amorphous after freeze-drying)

Made up to 100 g water as start formulation before freeze-drying

### Example 12

10⁶˜10¹² colony forming units Escherichia coli

1.0-5.0 g Mannitol

1.0 g Sucrose (amorphous after freeze-drying)

Made up to 100 g water as start formulation before freeze-drying

### Example 13

10⁶˜10¹² colony forming units Escherichia coli

1.0-5.0 g Mannitol

1.0 g Glucose (amorphous after freeze-drying)

Made up to 100 g water as start formulation before freeze-drying

### Example 14

10⁶˜10¹² colony forming units Escherichia coli

2.0 g Mannitol

1.0 g Maltose (amorphous after freeze-drying)

Made up to 100 g water as start formulation before freeze-drying

### Example 15

10⁶˜10¹² colony forming units Escherichia coli

2.0 g Mannitol

1.0 g Lactose (amorphous after freeze-drying)

Made up to 100 g water as start formulation before freeze-drying

It was found that Escherichia coli in Comparative Example 5 had less than 1% viability after the freeze drying process. In comparison Escherichia coli of Examples 11 to 15 according to the invention had a viability after lyophilisation of 60%.

In Examples 11a, 12a, 13a, 14a and 15a, each formulation was prepared in a manner identical to Examples 11 to 15 except that 30 mM thiourea was included as a free radical scavenger. Shelf stability for these formulations after freeze drying was improved from 30 days (for Comparative Example 5) to 200 days (measured as the time to lose 1 log viability) as measured by titres expressed as the number of colony forming units (cfu) per ml of bacterial suspension as plated using solid agar plates.

### EXAMPLE 16 TO 18

The viability of Salmonella typhimurium (live bacterium for diagnostic use) after freeze drying was measured for formulations according to the invention and for a comparative formulation. Measurement of viability was by titres expressed as the number of colony forming units (cfu) per ml of bacterial suspension as plated using solid agar plates.

The formulations set out below were prepared using the freeze drying cycle set out in Method Example 1:

### Comparative Example 6

10⁶˜10¹¹ colony forming units Salmonella typhimuriumi

5.0 g Mannitol

Made up to 100 g water as start formulation before freeze-drying

### Example 16

10⁶˜10¹¹ colony forming units Salmonella typhimuriumi

5.0 g Mannitol

1.0 g Sucrose (amorphous after freeze-drying

Made up to 100 g water as start formulation before freeze-drying

### Example 17

10⁶˜10¹¹ colony forming units Salmonella typhimuriumi

5.0 g Mannitol

1.0 g Trehalose (amorphous after freeze-drying)

Made up to 100 g water as start formulation before freeze-drying

### Example 18

10⁶˜10¹¹ colony forming units Salmonella typhimuriumi

5.0 g Mannitol

1.0 g Lactose (amorphous after freeze-drying)

Made up to 100 g water as start formulation before freeze-drying

It was found that Salmonella typhimurium in Comparative Example 6 had less than 1% viability after the freeze drying process. In comparison Salmonella typhimurium of Examples 16 to 18 according to the invention had a viability after lyophilisation of 40%.

### EXAMPLE 19 TO 22

The viability of Lactobacillus acidophilus (live bacterium for silaging use) after freeze drying was measured for formulations according to the invention and for a comparative formulation. Measurement of viability was by titres expressed as the number of colony forming units (cfu) per ml of bacterial suspension as plated using solid agar plates.

The formulations set out below were prepared using the freeze drying cycle set out in Method Example 1:

### Comparative Example 7

10⁶˜10¹² colony forming units Lactohacillus acidophilus

5.0 g Mannitol

Made up to 100 g water as start formulation before freeze-drying

### Example 19

10⁶˜10¹² colony forming units Lactobacillus acidophilus

10.0 g Mannitol

10.0 g Foetal Calf Serum (amorphous after freeze-drying)

Made up to 100 g water as start formulation before freeze-drying

### Example 20

10⁶˜10¹² colony forming units Lactobacillus acidophilus

5.0 g Mannitol

1.0 g Trehalose (amorphous after freeze-drying)

Made up to 100 g water as start formulation before freeze-drying

### Example 21

10⁶˜10¹² colony forming units Lactobacillus acidophilus

5.0 g Mannitol

1.0 g Lactose (amorphous after freeze-drying)

Made up to 100 g water as start formulation before freeze-drying

### Example 22

10⁶˜10¹² colony forming units Lactobacillus acidophilus

5.0 g Mannitol

1.0 g Sucrose (amorphous after freeze-drying)

Made up to 100 g water as start formulation before freeze-drying

It was found that Lactohacillus acidophilus in Comparative Example 7 had less than 1% viability after the freeze drying process. In comparison Lactobacillus acidophilus of Example 19 according to the invention had a viability after lyophilisation of 60 and the Lactobacillus acidophilus of Examples 20 to 22 according to the invention had a viability after lyophilisation of 40%.

### EXAMPLES 23 TO 26

The infectivity of influenza virus strain WSN (live, attenuated vaccine) after freeze drying was measured for formulations according to the invention and for a comparative formulation. Measurement of infectivity was expressed as plaque forming units (pfu per ml where 1 pfu=one lesion termed as ‘plaque’) in a chick embryo cell monolayer (defined as ‘sheet’) before and after freeze drying.

The formulations set out below were prepared using the freeze drying cycle set out in Method Example 1:

### Comparative Example 8

10⁸˜10¹¹ plaque forming units Influenza Virus strain WSN

1.0 g Sodium Chloride

Made up to 100 g water as start formulation before freeze-drying

### Example 23

10⁸˜10¹¹ plaque forming units Influenza Virus strain WSN

2.0 g Sodium Chloride

1.0 g Human Serum Albumin (amorphous after freeze-drying)

1.0 g Calcium Lactobionate

20 ml Chick Allantoic Fluid

Made up to 100 g water as start formulation before freeze-drying

### Example 24

10⁸˜10¹¹ plaque forming units Influenza Virus strain WSN

1.0 g Sodium Chloride

1.0 g Lactose (amorphous after freeze-drying)

2.0 g Dextran (mw 110,000, amorphous after freeze-drying)

Made up to 100 g water as start formulation before freeze-drying

### Example 25

10⁸˜10¹¹ plaque forming units Influenza Virus strain WSN

1.0 g Sodium Chloride

1.0 g Lactose (amorphous after freeze-drying)

1.0 g Sodium Monoglutamate (Maillard Reaction inhibitor)

Made up to 100 g water as start formulation before freeze-drying

### Example 26

10⁸˜10¹¹ plaque forming units Influenza Virus strain WSN

1.0 g Sodium Chloride

1.0 g Lactose (amorphous after freeze-drying)

1.0 g Ascorbic Acid (Antioxidant)

Made up to 100 g water as start formulation before freeze-drying

It was found that the influenza virus strain WSN in Comparative Example 8 had less than 1% infectivity after freeze drying. In comparison influenza virus strain WSN of Example 23 according to the invention had an infectivity after lyophilisation of 70% infectivity and the influenza virus strain WSN of Example 24 according to the invention had an infectivity after lyophilisation of 40%. The shelf stability of Examples 24 and 25 was improved one from a log loss of infectivity in approximately 40 days to a one loss of infectivity in greater than 1000 days as measured as plaque forming units (pfu per ml where pfu=one lesion termed ‘plaque’) in a chick embryo cell monolayer (defined as ‘sheet’) before and after freeze-drying (1 log loss=arithmetic loss of 90%) compared to Comparative Example 8.

### EXAMPLE 27

The activity of phage (p174 (phage for therapeutic or diagnostic use) after freeze drying was measured for formulations according to the invention and for a comparative formulation. Measurement of activity was expressed as plaque forming units (pfu per ml where 1 pfu=one lesion termed as ‘plaque’) in a Escherichia coli bacteria cell culture (defined as ‘sheet’) before and after freeze drying.

The formulations set out below were prepared using the freeze drying cycle set out in Method Example 1

### Comparative Example 9

10¹⁰ plaque forming units phage (p174

0.9 g Sodium Chloride

Made up to 100 g water as start formulation before freeze drying

### Example 27

10¹⁰ plaque forming units phage p174

0.9 g Sodium Chloride

0.1 g Serum Albumin

0.1 g Dextran Polymer

0.1 g Sucrose

Made up to 100 g water as start formulation before freeze drying

It was found that the phage p174 in Comparative Example 9 had less than 1% activity after freeze drying. In comparison phage p174 of example 27 according to the invention had an activity of 60%

### EXAMPLE 28

Examples 1 to 27 above demonstrate that to maintain the biological activity/viability of a formulation containing a sensitive active material during lyophilisation, it is necessary to include a component that is either induced into or retains an amorphous state during freeze-drying. However, it is known that freeze-dried, amorphous materials will absorb moisture from its environment leading to deterioration in the physical properties and often leading to the amorphous matrix become ‘sticky’ and unsuitable for further processing (such as de-aggregation, mixing, milling, dispensing and/or packaging etc.). Absorption of moisture will also adversely affect product storage stability. It is for this reason that freeze dried formulations of sensitive active materials are usually freeze dried in vials or other containers that can be sealed in the freeze dryer prior to exposure to an ambient environment. In contrast, formulations retaining a crystalline nature during the freeze drying process, while being ineffective at maintaining properties such as biological activity, possess stable physical properties, do not absorb appreciable quantities of moisture and, therefore, are highly shelf stable.

This is demonstrated in the example below. The following four solutions containing trehalose (known to be amorphous in nature following freeze drying) and mannitol (known to retain its crystallinity during freeze drying) were prepared and freeze dried in vials.

### COMPARATIVE EXAMPLE 10

| | |
|---|---|
| Mannitol | 200 mg |
| Water to | 2 mL |

Providing a 10% w/v mannitol solution

### COMPARATIVE EXAMPLE 11

| | |
|---|---|
| Mannitol | 20 mg |
| Water to | 2 mL |

Providing a 1% w/v mannitol solution

### COMPARATIVE EXAMPLE 12

| | |
|---|---|
| Trehalose | 200 mg |
| Water to | 2 mL |

Providing a 10% w/v trehalose solution

### COMPARATIVE EXAMPLE 13

| | |
|---|---|
| Trehalose | 20 mg |
| Water to | 2 mL |

Providing a 1% w/v trehalose solution

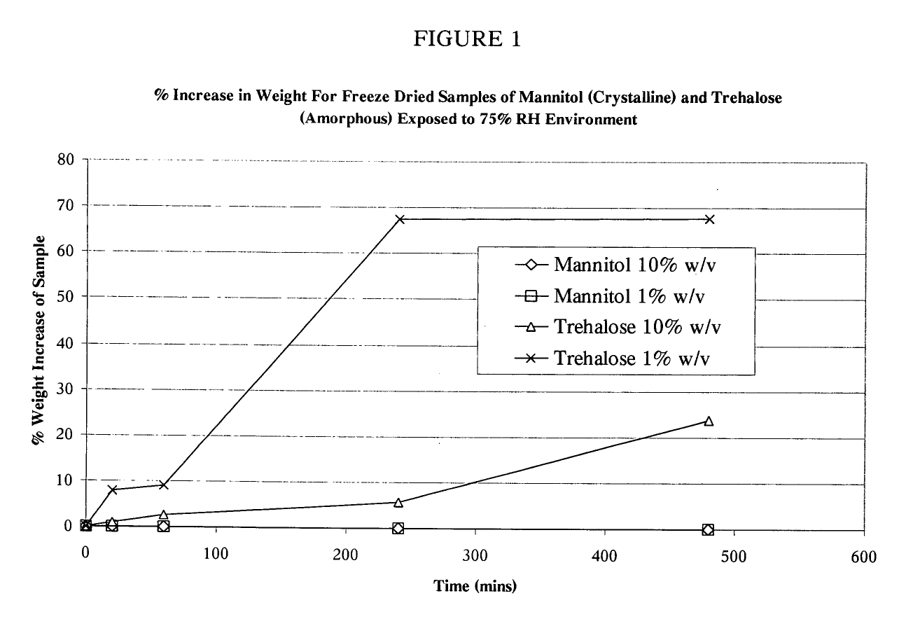

Following lyophilisation the resultant freeze dried matrices were exposed to a 75% RH (Relative Humidity) atmosphere for a period of 8 hours. At intervals over this period, moisture uptake of each sample was measured gravimetrically and calculated as a percentage weight increase of the sample. It is clear from FIG. 1 that while there is significant moisture uptake in the amorphous (trehalose) samples, there is no appreciable moisture uptake in the crystalline (mannitol) samples. The moisture uptake in the amorphous samples was accompanied by a deterioration in the physical quality of these samples.

However, by utilisation of a formulation containing a sensitive active material that comprises excipients with a crystalline/amorphous character according to the invention, it is possible both to retain the biological activity/viability of the sensitive active material (as seen in previous examples) and achieve stable physical properties that do not appreciably take up moisture. It was surprisingly found that Examples 1 to 27 of the invention had a substantially reduced moisture take up such that the increase in weight was of each sample was less than 3% by weight after exposure to a high (75%) relative humidity environment for eight hours.

1. A powdered formulation which is a freeze-dried mixture of a sensitive active material and an excipient comprising:
from 0.01 preferably from 0.1, more preferably from 0.5 to 50% by wt of the sensitive active material,
from 50 to 99.99, preferably to 99.9, more preferably to 99.5% by wt of the excipient, wherein of at least 0.1% by wt of the mixture is in an amorphous state.

2. A formulation according to claim 1, of from 0.1, preferably from 0.5, more preferably from 1 to 50% by wt of the freeze-dried mixture in an amorphous state.

3. A formulation according to claim 1, comprising:
from 0.01, preferably from 0.1, more preferably from 0.5 to 50% by wt of sensitive active material in an amorphous state,
from 50 to 99.99, preferably to 99.9, more preferably to 99.5% by wt of excipient in crystalline state,
0-5% by wt of excipient in an amorphous state.

4. A formulation according to claim 1, comprising:
from 0.01, preferably from 0.1, more preferably from 0.5 to 50% by wt of sensitive active material in a crystalline state,
from 50 to 99.89, preferably to 99.8, more preferably to 99.4% by wt of excipient in crystalline state, and
0.1-5% by wt of excipient in an amorphous state.

5. A formulation according to claim 1, comprising:
from 0.01, preferably from 0.1, more preferably from 0.5 to 25% by wt of an amorphous or a crystalline state of sensitive active material,
from 75 to 99.49, preferably to 99.4, more preferably to 99% by wt of a crystalline state excipient, and
0.5-5% by wt of excipient in an amorphous state.

6. A formulation according to claim 1 in which a saccharide is used to provide an excipient in an amorphous state.

7. A formulation according to claim 1 in which a sugar alcohol is used to provide an excipient in a crystalline state.

8. A formulation according to claim 1 wherein the formulation additionally comprises from 0.1 to 10% by wt (preferably from 1 to 10% by wt) of additive/stabilizer.

9. A formulation as defined in claim 8 wherein the additive/stabilizer is an antioxidant, a free radical scavenger and/or a Maillard reaction suppresser.

10. A formulation according to claim 1 wherein the sensitive active material is a labile organic and/or inorganic molecule, a biopolymer, a polypeptide, protein, enzyme, hormone, vitamin, antibiotic, polysaccharide, lipid, killed or live whole live cell.

11. A formulation according to claim 10 wherein the sensitive active material is a virus (including phage), bacterium, fungus and/or eukaryote.

12. A formulation according to claim 1 which has a stable crystalline/amorphous matrix.

13. A formulation according to claim 1 which has a substantially reduced hygroscopicity.

14. A formulation according to claim 1 which has a hygroscopicity of less than 5% by weight, preferably less than 3% by weight, more preferably less than 2% by weight, wherein the hygroscopicity is measured by the percentage increase in the weight of the formulation after 8 hours in a 75% relative humidity environment.

15. A dosage form comprising a formulation according to claim 1.

16. A dosage form according to claim 15 which is a container which comprises the formulation or an article which has been formed from the formulation.

17. A method of preparing a powdered formulation which comprises forming a mixed solution of sensitive active material and excipient(s) containing:
from 0.01 preferably from 0.1, more preferably from 0.5 to 50% by wt of the sensitive active material,
from 50 to 99.99, preferably to 99.9, more preferably to 99.5% by wt of the excipient, and freeze-drying the solution so that at least 0.1% by wt of the freeze-dried blend is in an amorphous state.

18. A method according to claim 17 in which the active material freeze dries to a crystalline state and the mixed solution contains:
from 0.01, preferably from 0.1, more preferably from 0.5 to 50% by wt of sensitive active material in amorphous state,
from 50 to 99.99, preferably to 99.9, more preferably to 99.5% by wt of excipient in crystalline state,
0.1-5% by wt of excipient which freeze dries to an amorphous state.

19. A method according to claim 17 in which the active material freeze dries to an amorphous state and the mixed solution contains:
from 0.01, preferably from 0.1, more preferably from 0.5 to 50% by wt of sensitive active material in crystalline state,
from 50 to 99.89, preferably to 99.8, more preferably to 99.4% by wt of excipient in crystalline state,
0-5% by wt of excipient which freeze dries to an amorphous state.

20. A method according to claim 17, in which the mixed solution contains:
from 0.01, preferably from 0.1, more preferably from 0.5 to 25% by wt of amorphous or crystalline state of sensitive active material,
from 75 to 99.49, preferably to 99.4, more preferably to 99% by wt of crystalline state excipient, 0.1-5% by wt of excipient which freeze dries to an amorphous state.

21. A method according to claim 17 in which a sugar is used to provide an excipient in amorphous state.

22. A method according to claim 17 in which a sugar alcohol is used to provide an excipient in crystalline state.

23. A method of medical treatment which method comprises supplying to a human or animal patient a therapeutically effective amount of a formulation according to claim 1.

24. A method of medical treatment which method comprises supplying to a human or animal patient a therapeutically effective amount of a dosage form according to claim 15.

25. A method of reducing the hygroscopicity of a freeze dried formulation which is a freeze-dried mixture of a sensitive active material and an excipient containing:
from 0.01 preferably from 0.1, more preferably from 0.5 to 50% by wt of the sensitive active material, and
from 50 to 99.99, preferably to 99.9, more preferably to 99.5% by wt of the excipient,
wherein the method comprises the step of including in the formulation at least 0.1% by wt of a sensitive active material and/or an excipient in an amorphous state.
